(19) United States
(12) Patent Application Publication
(10) Pub. No.: US 2010/0190729 A1
(43) Pub. Date: Jul. 29, 2010

(54) Composition for treating atopic dermatitis comprising hirsutanonol or oregonin as an active ingredient

(75) Inventors: Min Won Lee, Seoul (KR); Young Wook Choi, Seoul (KR); Seong Jun Seo, Anyang-si (KR); Do Ik Lee, Seoul (KR); Hyoweon Bang, Seoul (KR); Chung Soo Lee, Seoul (KR); Jong Chan Lee, Seoul (KR); Soon-Chul Myung, Sungnam-si (KR); Mi-Kyung Lee, Seoul (KR); Seong Soo Joo, Suwon-si (KR); Sun Eun Choi, Seoul (KR)

Correspondence Address: CLARK & ELBING LLP, 101 FEDERAL STREET, BOSTON, MA, 02110, US

(73) Assignee: CHUNG-ANG University Industry Academic Cooperation Foundation, Seoul (KR)

(21) Appl. No.: 12/454,552

(22) Filed: May 19, 2009

(30) Foreign Application Priority Data
Jan. 28, 2009 (KR) 10-2009-0006636

(51) Int. Cl. A61K 31/7034; A61K 31/12; A61P 17/02; A61P 37/06; A61Q 90/00

(52) U.S. Cl. 514/25; 514/679

(57) ABSTRACT

The present invention relates to a composition for treating atopic dermatitis comprising hirsutanonol as an active ingredient. Hirsutanonol or oregonin as the active ingredient of the present composition decreases the number of eosinophil increased in atopic dermatitis and regulates expression amounts of immune regulatory cytokines, IL-4, IL-5, IL-10 and IL-13 associated with atopic dermatitis. In addition, hirsutanonol and oregonin increase MBD (mouse beta-defensin)-1, MBD-2 and MBD-3 expression and decrease COX-2 and iNOS expression in mouse animal model of atopic dermatitis. Hirsutanonol and oregonin as the active ingredient of the present composition could be effectively used in drugs, cosmetics and foods for treating atopic dermatitis.

### BACKGROUND OF THE INVENTION

1. Field of the Invention

The present invention relates to a composition for treating atopic dermatitis comprising hirsutanonol or oregonin as an active ingredient.

2. Background of the Invention

Hirsutanonol belongs to compounds having a structure of diarylheptanoid extracted from stem bark of the genus Alnus. Asakawa et al. isolated four kinds of compounds (e.g., alnustone) involving a new structure of diarylheptanoid extracted from flower of A. pendula in 1972 and Miyake et al. in 1973 reported that hirsutanonol and hirsutenone were isolated from green stem bark of A. hirsute (Suga, T., Iwata, N. and Asakaw, Y.: Chemical constituents of male flower of Alnus pendula. Bull. Chem. Soc. Jap., 45, 2058-2060, 1972).

Doug et al. discovered that diarylheptanoid plays a critical role in strong prevention of platelet coagulation in 1998 (Doug, H., Chen, S. X., Xu, H. X., Kadota, S, and Namba, T.: A new antilplatelet diarylheptanoid from Alpinia blepharocalyx. J. Nat. Prod., 61, 142-144, 1998).

In addition, Lee et al. reported diarylheptanoid as a new PKC alpha inhibitor in 1998 (Lee, K. K., Bahler, B. D., Hofmann, G. A., Mattern, M. R., Johnson, R. K. and Kingston, D. G. I.: Isolation and structure elucidation of new PKCα inhibitor from Pinus flexilis. J. Nat. Prod., 61, 1407-1409, 1998).

Surh et al. in 1999 and Ishida et al. in 2000 also found that diarylheptanoids have an antitumor-promoting potential (Chun, K.-S., Sohn, Y.-S., Kim, H.-S., Kim, O.-H., Park, K.-K., Lee, J.-M., Lee, J., Lee, J.-Y., Moon, A., Lee, S.-S, and Surh, Y.-J.: Antitumor promoting potential of naturally occurring diarylheptanoids structurally related to curcumin, Mutation Research, 428, 49-57, 1999; Ishida, J. Kozuka, M., Wang, H.-K., Konoshima, T., Tokuda, H., Okuda, M., Mou, X. Y., Nishino, H., Sakurai, N., Lee, K.-S, and Nagai, M.: Antitumor-promoting effects of cyclic diarylheptanoids on Epstein-Barr virus activation and two-stage mouse skin carcinogenesis, Cancer Letters, 159, 135-140, 2000).

The term “atopy” refers to a meaning to be “extraordinary” or “inappropriate” on etymology. Atopic dermatitis is a chronic inflammatory disease which repeats improvement and aggravation after its attack in babyhood or infancy, and is diagnosed according to three features of individual or familial atopy, severe itching and eczema. In addition, atopic dermatitis could be worsen by infection, mental stress, changes of season and weather, stimulation and allergy.

The etiology of atopic dermatitis remains to be clearly elucidated, but according to recent researches, the reasons of attack to induce atopic dermatitis are as follows: (a) hypersensitive response caused from increase of IgE antibody, (b) functional defect by irregular differentiation of T lymphocyte which is caused from reduction of cell-mediated immune response, and (c) blocking of adrenal receptor present in the skin. Therefore, atopic dermatitis has been thought to be a hereditary disorder generated by immunological abnormality.

In general, humectant to preserve the moisture on the skin and steroid hormone (e.g., local antenatal corticosteroid) to alleviate inflammation response are simultaneously treated in most dermatologic clinic for the treatment and management of atopic dermatitis. When a local antenatal corticosteroid is used for a long period, it causes a serious problem to produce various side effects on skin such as dermal atrophy, vasodilatation, depigmentation and striae distensae. Therefore, it has been endeavored to develop a raw material or drug having anti-inflammatory efficacy for treating atopic dermatitis without the above-mentioned side effects.

It has been disclosed that hirsutanonol compounds have anti-cancer function and anti-oxidative activity, but their physiological effects on atopic dermatitis have not been reported yet.

Throughout this application, various publications and patents are referred and citations are provided in parentheses. The disclosures of these publications and patents in their entities are hereby incorporated by references into this application in order to fully describe this invention and the state of the art to which this invention pertains.

### DETAILED DESCRIPTION OF THE INVENTION

The present inventors have made intensive researches to develop a compound for treating atopic dermatitis from natural extracts. As results, we have discovered that hirsutanonol and oregonin, diarylheptanoid compounds among compounds contained in stem bark extracts of Alnus japonica, regulates an expression level of the immune cytokines associated with atopic dermatitis and also treats atopic dermatitis or alleviates a symptom of atopic dermatitis through its regulatory activities.

Accordingly, it is an object of this invention to provide a composition for treating atopic dermatitis, comprising hirsutanonol or oregonin as an active ingredient.

Other objects and advantages of the present invention will become apparent from the following detailed description together with the appended claims and drawings.

In one aspect of this invention, there is provided a pharmaceutical composition for treating or preventing atopic dermatitis, comprising: (a) a therapeutically effective amount of an isolated hirsutanonol or oregonin; and (b) a pharmaceutically acceptable carrier.

In another aspect of this invention, there is provided a cosmetic composition for relieving atopic dermatitis, comprising: (a) a cosmetically effective amount of an isolated hirsutanonol or oregonin; and (b) a cosmetically acceptable carrier.

In still another aspect of this invention, there is provided a functional food composition for relieving atopic dermatitis, comprising an isolated hirsutanonol or oregonin as an active ingredient.

In still another aspect of this invention, there is provided a method for treating atopic dermatitis in a subject suffering from atopic dermatitis, which comprises contacting said subject with a composition comprising a therapeutically effective amount of an insolated hirsutanonol or oregonin or administering said composition to said subject.

In still another aspect of this invention, there is provided a method for relieving a symptom of atopic dermatitis in a subject suffering from atopic dermatitis, which comprises contacting said subject with a composition comprising an insolated hirsutanonol or oregonin or administering said composition to said subject.

The present inventors have made intensive researches to develop a compound for treating atopic dermatitis from natural extracts. As results, the inventors have discovered that hirsutanonol or oregonin, diarylheptanoid compounds among compounds contained in stem bark extracts of Alnus japonica, regulates an expression level of the immune cytokines associated with atopic dermatitis and also treats atopic dermatitis or alleviates a symptom of atopic dermatitis through its regulatory activities.

Hirsutanonol used as the active ingredient of the present composition is a compound represented by the following formula 1.

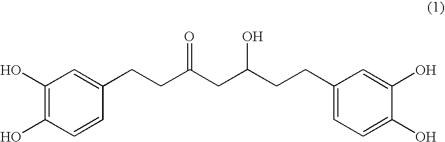

Oregonin used as the other active ingredient of the present composition is a compound represented by the following formula 2.

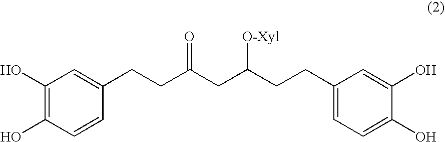

wherein ‘Xyl’ represents xylose in the formula 2.

Hirsutanonol or oregonin as the active ingredient of the present composition could be obtained from natural products, for example stem bark extracts of Alnus japonica. It is well known to those skilled in the art that chemically synthesized hirsutanonol or oregonin compound could also have the same effect as much as the one obtained from extracts have.

Stem bark extracts of Alnus japonica in this invention may be prepared according to a conventional method known in the art, for example utilization of conventional solvent under conditions of typical temperature and pressure. In general, the conventional solvent involved in extraction process is used as the extraction solvent for isolating stem bark extracts of Alnus japonica, and preferably is selected from the groups consisting of water, anhydrous or hydrated lower alcohol containing 1 to 4 carbon atoms, acetone, ethylacetate, butylacetate and 1,3-butylene glycol.

Hirsutanonol or oregonin as an active ingredient of the present composition decreases the number of eosinophil increased in atopic dermatitis and regulates an expression level of immune regulatory cytokines, IL-4, IL-5, IL-10 and IL-13 which are associated with atopic dermatitis. In addition, hirsutanonol and oregonin increase MBD (mouse beta-defensin)-1, MBD-2 and MBD-3 expression and decrease COX-2 and iNOS expression in mouse animal model of atopic dermatitis.

The composition of this invention may be provided as a pharmaceutical composition. The term “pharmaceutically effective amount” refers to an amount enough to show and accomplish efficacies and activities of the compound of this invention for treating atopic dermatitis. The pharmaceutical composition of this invention includes a pharmaceutically acceptable carrier besides the active ingredient compound.

The pharmaceutically acceptable carrier contained in the pharmaceutical composition of the present invention, which is commonly used in pharmaceutical formulations, but is not limited to, includes lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methylcellulose, methylhydroxy benzoate, propylhydroxy benzoate, talc, magnesium stearate, and mineral oils. The pharmaceutical composition according to the present invention may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

A suitable dosage amount of the pharmaceutical composition of the present invention may vary depending on pharmaceutical formulation methods, administration methods, the patient's age, body weight, sex, pathogenic state, diet, administration time, administration route, an excretion rate and sensitivity for a used pharmaceutical composition. Preferably, the pharmaceutical composition of the present invention may be administered with a daily dosage of 0.001-200 mg/kg (body weight).

The pharmaceutical composition according to the present invention may be administered orally or parenterally, and preferably, administered parenterally, e.g., by intravenous, intraperitoneal, intramuscular, intra-abdominal or transdermal.

According to the conventional techniques known to those skilled in the art, the pharmaceutical composition according to the present invention may be formulated with pharmaceutically acceptable carrier and/or vehicle as described above, finally providing several forms including a unit dose form and a multi-dose form. Non-limiting examples of the formulations include, but not limited to, a solution, a suspension or an emulsion in oil or aqueous medium, an elixir, a powder, a granule, a tablet and a capsule, and may further comprise a dispersion agent or a stabilizer.

According to a preferable embodiment, the pharmaceutical composition of the present invention is formulated for a skin application. The formulation for a skin application is not particularly limited and preferably includes a powder, a gel, an ointment, a cream, a fluid or an aerosol.

The composition of this invention may be provided as a cosmetic composition. The term used herein “cosmetically effective amount” refers to an amount enough to accomplish efficacies on improvements in atopic dermatitis described hereinabove.

The pharmaceutical composition of this invention includes a cosmetically acceptable carrier besides the active ingredient compound.

The cosmetic compositions of this invention may be formulated in a wide variety of forms, for example, including a solution, a suspension, an emulsion, a paste, a gel, a cream, a lotion, a powder, a soap, a surfactant-containing cleanser, an oil, a powder foundation, an emulsion foundation, a wax foundation and a spray but not limited to. Specifically, the cosmetic compositions of this invention may be formulated in the form of skin softener, nutrient liquid, nutrient cream, massage cream, essence, eye cream, cleansing cream, cleansing foam, cleansing water, pack, spray or powder.

Where the cosmetic composition is in the form of paste, cream or gel, it may comprise animal and vegetable fats, waxes, paraffins, starch, tragacanth, cellulose derivatives, polyethylene glycols, silicones, bentonites, silica, talc, zinc oxide or mixture of these substances.

In the formulation of powder or spray, it may comprise lactose, talc, silica, aluminum hydroxide, calcium silicate, polyamide powder and mixtures of these substances. Spray may additionally comprise the customary propellants, for example, chlorofluorohydrocarbons, propane/butane or dimethyl ether.

The formulation of solution and emulsion may comprise solvent, solubilizer and emulsifier, for example water, ethanol, isopropanol, ethyl carbonate, ethyl acetate, benzyl alcohol, benzyl benzoate, propylene glycol, 1,3-butylglycol, oils, glycerol fatty esters, polyethylene glycol and fatty acid esters of sorbitan.

The formulation of suspension may comprise liquid diluents, for example water, ethanol or propylene glycol, suspending agents, for example ethoxylated isosteary alcohols, polyoxyethylene sorbitol esters and poly oxyethylene sorbitan esters, micocrystalline cellulose, aluminum metahydroxide, bentonite, agar and tragacanth or mixtures of these substances.

The formulation of cleansing compositions with surfactant may comprise aliphatic alcohol sulfate, aliphatic alcohol ether sulfate, sulfosucinnate monoester, isothinate, imidazolium derivatives, methyltaurate, sarcocinate, fatty acid amide ether sulfate, alkyl amido betain, aliphatic alcohol, fatty acid glyceride, fatty acid diethanolamide, vegetable oil, lanoline derivatives, ethoxylated glycerol fatty acid ester or mixtures of these ingredients.

Furthermore, the cosmetic compositions of this invention may contain auxiliaries as well as compounds as active ingredients and carriers. The non-limiting examples of auxiliaries include antioxidants, stabilizers, solubilizers, vitamins, colorants, odor improvers or mixtures of these substances.

The composition of the present invention may be provided as a food composition, particularly a functional food composition. The functional food composition of the present invention may be formulated in a wide variety of forms, for example, including proteins, carbohydrates, fatty acids, nutrients and seasoning agents. In the formulation of drinking agent, it may further include a flavoring agent or natural carbohydrates. For instance, natural carbohydrate may include monosaccharides (e.g., glucose, fructose, etc.); disaccharides (e.g., maltose, sucrose, etc.); oligosaccharides; polysaccharides (e.g., dextrin, cyclodextrin, etc.); and sugar alcohols (e.g., xylitol, sorbitol, erythritol, etc.). The formulation of flavoring agent may use natural flavoring agents (e.g., thaumatin, stevia extract, etc.) and synthetic flavoring agents (e.g., saccharine, aspartame, etc.). The food composition of the present invention may be much effectively utilized to improve atopic dermatitis.

The features and advantages of the present invention will be summarized as follows:

(i) The present invention provides a new use of known compounds of hirsutanonol and oregonin for treating atopic dermatitis or alleviating a symptom of atopic dermatitis.

(ii) Hirsutanonol and oregonin as the active ingredient of the present composition decrease the number of eosinophil increased in atopic dermatitis and regulates an expression level of immune regulatory cytokines, IL-4, IL-5, IL-10 and IL-13 which are associated with atopic dermatitis.

(iii) Hirsutanonol and oregonin as an active ingredient of the present composition increase an expression level of MBD (mouse beta-defensin)-1, MBD-2 and MBD-3 and decrease an expression level of COX-2 and iNOS in mouse animal model of atopic dermatitis.

(iv) Hirsutanonol and oregonin as an active ingredient of the present composition could be effectively used in drugs, cosmetics and foods for treating or alleviating atopic dermatitis.

The present invention relates to a composition for treating atopic dermatitis or relieving a symptom of atopic dermatitis comprising hirsutanonol as an active ingredient. Hirsutanonol or oregonin as the active ingredient of the present composition decreases the number of eosinophil increased in atopic dermatitis and regulates an expression level of immune regulatory cytokines, IL-4, IL-5, IL-10 and IL-13 which are associated with atopic dermatitis. In addition, hirsutanonol and oregonin increase an expression level of MBD (mouse beta-defensin)-1, MBD-2 and MBD-3 and decrease an expression level of COX-2 and iNOS in mouse animal model of atopic dermatitis. Hirsutanonol and oregonin as an active ingredient of the present composition could be effectively used in drugs, cosmetics and foods for treating atopic dermatitis.

### BRIEF DESCRIPTION OF THE DRAWINGS

FIG. 1 schematically represents a process to extract and purify hirsutanonol and oregonin from the stem bark of Alnus japonica.

FIG. 2a represents ¹H-NMR and ¹³C-NMR spectra of oregonin purified from the stem bark of Alnus japonica.

FIG. 2b represents ¹H-NMR and ¹³C-NMR spectra of hirsutanonol purified from the stem bark of Alnus japonica.

FIG. 2c represents a result of performing HPLC (high performance liquid chromatography) on oregonin.

FIG. 3 is results of a LDH (lactate dehydrogenase) assay to analyze a cytotoxicity of oregonin to Jurkat cells. Jurkat cells were incubated with oregonin at 37° C. for 24 hrs in a humidified atmosphere containing 5% CO₂. GO: oregonin.

FIG. 4 is a graph representing a removal efficiency of nitric oxide (NO) by oregonin. 200 μM SNAP(S-nitrosol-N-acetylpenicillamine) solution as a NO donor, 50 μM Vitamin C solution and 10 μM genistein solution were prepared. The final reaction volume was adjusted to 150 μl and incubated at 37° C. for 0-8 hrs. 50 μl of each sample was mixed with the same volume of Griess reagent and then the absorbance was measured at 540 nm.

FIG. 5 represents a result of western blotting to test BSA degradation by oregonin. The hydroxyl radical-mediated oxidation experiments were carried out by metal-catalyzed reactions. BSA (bovine serum albumin) was dissolved in 150 mM phosphate buffer (pH 7.3) to a final concentration of 0.5 mg/ml. BSA solution was incubated with 100 μM Cu²⁺ and 2.5 mM hydroperoxide (H₂O₂) for 2 hrs in the presence or absence of oregonin. 50 μg/ml of ascorbic acid was directly dissolved in 1×PBS and served as a control (an anti-oxidant). After incubation in an open tube, the reaction was kept to stand in a 37° C. shaking water bath. Each experimental group was loaded on 10% SDS-PAGE and stained with 0.01% Coomassie blue.

FIG. 6 is the experimental results showing an inhibitory effect of oregonin on IL-2 and IL-4 expression. Panel A and B represent the amounts of IL-2 detected using ELISA microplate reader in 96-well plate. For optimal T cell activation, Jurkat cells were inoculated on a 96-well plate coated with anti-CD3 and anti-CD26 mAb, followed by addition of oregonin. Panel C represents the result detecting expression amounts of IL-4 mRNA in RBL-2H3 cells using RT-PCR. PCR primers were prepared according to the manufacturer's instruction: sense: 5′-cacggatgtaacgacagccctctg-3′, anti-sense: 5′-gcgtggactcattcacggtgcagc-3′.

FIG. 7 is a graph measuring the inhibitory effect of hirsutanonol on cell proliferation. Jurat cells were seeded into a 96 well plate at a concentration of 1×10⁴ cells per well, and cultured in 2% FBS DMEM with or without hirsutanonol. Cell numbers were counted in triplicate wells by measuring the absorbance of reduced WST-8 (Dojindo, Japan) at 450 nm.

FIG. 8 is the experimental results showing the inhibitory effect of hirsutanonol on IL-2 expression. Murine splenocytes were grown on 96 well plate at a concentration of 1.5×10⁴ cells per well. For T cell activation, CsA (50 nM) and hirsutanonol were added into a well added with anti-CD3 mAb and into all of other wells. Cells were incubated for 72 hrs and then ELISA was carried out using the supernatants collected from each well.

FIG. 9 represents RT-PCR results showing an inhibitory effect of hirsutanonol on Th1 and Th2 cytokine expression. Murine splenocytes were cultured on 12 well plate (RT-PCR) at a concentration of 1.5×10⁴ cells per well. For T cell activation, CsA (50 nM) and hirsutanonol were added into a well added with anti-CD3 mAb and all of other wells. Cells were incubated for 24 hrs and then ELISA was carried out. GAPDH was served as an internal control.

FIG. 10 represents experimental results of β-hexosaminidase assay for hirsutanonol. The β-hexosaminidase (beta-hex) assay was performed according to the standard protocol of RBL-2H3 cell line. Suitable supernatant was collected and beta-hex activity was measured by incubating with a beta-hex substrate (p-nitrophenyl N-acetyl-beta-D-glucosaminide; Sigma) colored at 405 nm by cutting. As shown by the measured absorbance, it has been demonstrated that the amounts of beta-hex release are different each other.

FIG. 11 represents a result where anti-oxidative activities of hirsutanonol were indirectly observed by determining whether the BSA degradation was protected. 50 μg of BSA (Sigma) was added to 37.5 mM phosphate buffers (pH 7.3) containing 100 μM Cu²⁺, 2.5 mM H₂O₂ and various amounts of hirsutanonol (1, 50, 100/200 μg/ml), and incubated at 37° C. for 2 hrs. Vitamin C (50 μg/ml) was served as a positive control. The reaction mixture was separated on 10% SDS gel and the protein bands were stained with 0.01% Coomassie blue. Vitamin C represents Vitamin C; M is a size marker.

FIG. 12 is a flow cytometry result analyzing the changes of CD25/CD69 expression induced by hirsutanonol. Splenocytes were stimulated by anti-CD3 mAb in the presence or absence of hirsutanonol. Each hirsutanonol and CsA was treated at concentrations of 50 and 100 μg/ml, and 50 nM. After incubation for 24 hrs, the percentage of cells expressing CD25 and CD69 were measured using flow cytometry.

FIG. 13a is photographs observing treatment effects on atopic dermatitis after injection of PBS into mouse in which the atopic dermatitis was induced as a negative control.

FIG. 13b is photographs showing treatment effects on atopic dermatitis after injection of 0.1% oregonin into mouse in which the atopic dermatitis was induced.

FIG. 13c is photographs showing treatment effects on atopic dermatitis after injection of 1% oregonin into mouse in which the atopic dermatitis was induced.

FIG. 13d is photographs showing treatment effects on atopic dermatitis after injection of dexamethasone into mouse in which the atopic dermatitis was induced as a positive control.

FIG. 13e is photographs showing treatment effects on atopic dermatitis after injection of the composition without the active ingredient into mouse in which the atopic dermatitis was induced as a negative control.

FIG. 13f is photographs showing treatment effects on atopic dermatitis after injection of the formulation containing 1% oregonin into mouse in which the atopic dermatitis was induced.

FIG. 13g is photographs showing treatment effects on atopic dermatitis after injection of the formulation containing plancol into mouse in which the atopic dermatitis was induced as a positive control.

FIGS. 14a and 14b are graphs showing the results of measuring the ratio and number of eosinophile in blood samples obtained from each atopic dermatitis induced NC/Nga mouse before or after injection of PBS or a composition without an active ingredient (Baseline) (negative control), dexamethasone (DEX) or plancol (PLA) (positive control), or 0.1% or 1% oregonin (ORE) (experimental group).

FIGS. 15a-15d are graphs representing the levels of IL-4, IL-5, IL-10 and IL-13 measured by ELISA in serum and lymph node of the blood and spleen samples obtained from each atopic dermatitis induced NC/Nga mouse before or after being injected with PBS or a composition without an active ingredient (Baseline) (negative control), dexamethasone (DEX) or plancol (PLA) (positive control), or 0.1% or 1% oregonin (ORE) (experimental group).

FIGS. 16a-16e are graphs showing the expression levels of MBD-1, MBD-2, MBD-3, COX-2 and iNOS measured by real-time PCR with total mRNAs obtained from each NC/Nga mouse skins before or after being injected with PBS or a composition without an active ingredient (Baseline) (negative control), dexamethasone (DEX) or plancol (PLA) (positive control), or 0.1% or 1% oregonin (ORE) (experimental group).

FIGS. 17a-17e are graphs showing the expression levels of MBD-1, MBD-2, MBD-3, COX-2 and iNOS measured by western blotting with total proteins obtained from each NC/Nga mouse skins before or after being injected with PBS or a composition without an active ingredient (Baseline) (negative control), dexamethasone (DEX) or plancol (PLA) (positive control), or 0.1% or 1% oregonin (ORE) (experimental group).

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Methods and Materials

### 1. Experimental Materials

The stem bark of Alnus japonica used for the extraction of hirsutanonol and oregonin was collected in Sudal mountain, dongjak-gu, Seoul on June, 2008 and confirmed by plant judgement.

### 2. Instruments and Reagents

The instruments and reagents used in the examples were as follows:

TABLE 1
Instruments and reagents used in this example

| | |
|---|---|
| Balance | Sartorius AC211S (Germany) |
| Centrifuge | Eppendorff 5415D (Germany) |
| Liquid chromatography | API 3000 triple quadrupole liquid |
| mass spectrometer | chromatography mass spectrometry (Canada) |
| ¹H-NMR spectrometer | Varian Gemini 2000, 300 MHz (USA) |
| | Bruker AMX-500, 500 MHz (Germany) |
| | Solvent: DMSO-d₆, D₂O, Acetone-d₆ |
| | Internal standard: TMS |
| ¹³C-NMR spectrometer | Varian Gemini 2000, 75 MHz (USA) |
| | Bruker AMX-500, 125 MHz (Germany) |
| | Solvent: DMSO-_{d6}, D₂O, Acetone-d₆ |
| | Internal standard: TMS |
| TLC Adsorbent | Kieselgel 60 F₂₅₄ (Merck, Germany) |
| TLC Solvent(v/v) | CHCl₃:MeOH:H₂O = 70:30:4 |
| | CHCl₃:MeOH:H₂O = 6:4:1 |
| | Benzene:Ethylformate:Formic acid = 1:7:1 |
| TLC Detection | Ethanolic-FeCl₃ solution |
| | 10%-H₂SO₄ in ethanol (heating) |
| | UV-lamp (254 nm) |
| Gels | Sephadex LH-20 (25-100 μm, |
| | Pharmacia, Sweden) |
| | MCI gel CHP 20P (75-150 μm, |
| | Mitsubishi, Japan) |

### 3. Preparation of Extracts and Isolation of Active Ingredients

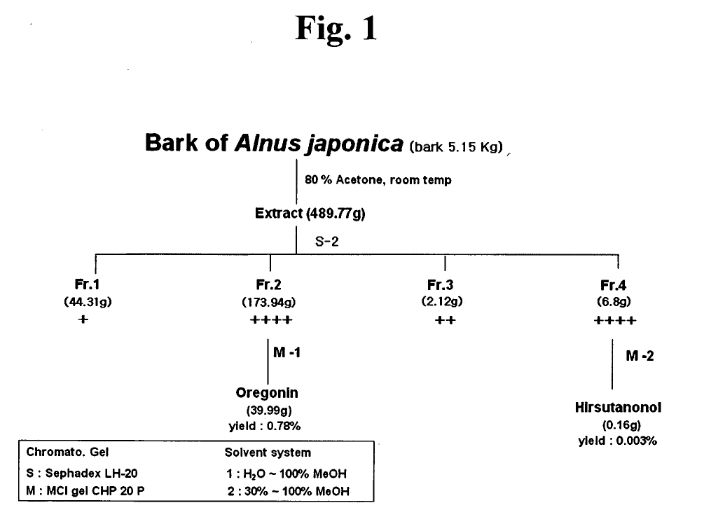

The fresh stem barks (5.15 kg) of Alnus japonica after harvest were extracted and filtered three times with 80% acetone at room temperature for 24 hrs. The extracted substances (489.77 g) were obtained by concentrating the extract solutions under reduced pressure and suspended in water. After filtration under reduced pressure, Sephadex LH-20 column chromatography of the aqueous portion was carried out. The solvent was increased in a linear gradient from 30% to 100% methanol by every 10% rise and divided into four sub-fractions by TLC method. MCl-gel CHP 20P column chromatography (0-400% methanol, gradient system) was performed on the fraction 2 (Fr. 2) where TLC response of oregonin and excellent DPPH activity was demonstrated. Finally, oregonin was extracted and purified in the amount of 39.99 g and its yield rate was 0.78%. In addition, MCl-gel CHP 20P column chromatography (30%→100% methanol, gradient system) was carried out on the fraction 4 (Fr. 4) in which TLC response of hirsutanonol and excellent DPPH activity was detected. Finally, hirsutanonol was extracted and purified in the amount of 0.16 g and its yield rate was 0.003%. In FIG. 1, the procedures of collecting extracted solution from the stem bark of Alnus japonica and purifying hirsutanonol and oregonin as an active ingredient from the extraction were schematically represented.

### 4. Characterization of the Chemical Structure

4.1. Oregonin

The isolated oregonin as an active ingredient was a form of amorphous brown powder. MS and NMR data was as follows.

[α]²⁰_{D}: −17.5° (c=1.0, MeOH)

Negative FAB MS: m/z 477 [M-H]⁻

¹H-NMR (300 MHz, Acetone-d₆+D₂O): δ 6.74-6.71 (4H in total, H-2′,2″, 5′,5″), 6.53-6.50 (2H in total, m, H-6″,6′), 4.31 (1H, d, J=7.8 Hz, xyl-1), 4.14 (1H, m, H-5), 3.86 (1H, dd, J=11.4, 6.1 Hz xyl-5e), 3.54 (1H, m, xyl-4), 2.83-2.52 (8H in total, H-1,2,4,7), 1.80-1.76 (2H in total, m, H-6)

¹³C-NMR (75 MHz, Acetone-d6+D₂O): described in the following Table 2.

4.2. Hirsutanonol

The isolated hirsutanonol as an active ingredient was a form of amorphous brown powder. MS and NMR data was as follows.

[α]²⁰_{D}: −25.2° (c=1.0, MeOH)

Negative FAB MS: m/z 345 [M-H]⁺

¹H-NMR (300 MHz, Acetone-d₆): δ 6.75-6.71 (4H in total, m, H-2′,2″, 5′,5″), 6.54-6.51 (2H in total, m, H-6′,6″), 4.06 (1H, m, H-5), 2.76-2.52 (8H in total, m, H-1,2,4,7), 1.72-1.65 (2H in total, m, H-6)

¹³C-NMR (75 MHz, Acetone-d₆+D₂O): described in the following Table 2.

TABLE 2
¹³C-NMR data of hirsutanonol and oregonin

| Carbon number | Oregonin | Hirsutanonol |
|---|---|---|
| Heptanes moiety | | |
| C-1 | 29.7 | 29.5 |
| C-2 | 46.1 | 42.6 |
| C-3 | 210.6 | 211.3 |
| C-4 | 48.2 | 50.7 |
| C-5 | 76.1 | 67.7 |
| C-6 | 38.3 | 40.0 |
| C-7 | 31.4 | 31.7 |
| Diphenyl moiety | | |
| C-1′ | 133.9 | 133.8 |
| C-1″ | 134.9 | 134.7 |
| C-2′ | 116.1 | 116.1 |
| C-2″ | 116.2 | 116.2 |
| C-3′ | 145.9 | 145.8 |
| C-3″ | 145.9 | 145.8 |
| C-4′ | 144.0 | 143.8 |
| C-4″ | 144.3 | 144.0 |
| C-5′ | 116.4 | 116.2 |
| C-5″ | 116.5 | 116.3 |
| C-6′ | 120.5 | 120.3 |
| C-6″ | 120.4 | 120.2 |
| Sugar moiety | | |
| C-1 | 104.0 | |
| C-2 | 74.6 | |
| C-3 | 77.5 | |
| C-4 | 70.8 | |
| C-5 | 66.6 | |
| C-6 | (xyl) | |

* 75 MHZ (Acetone-d₆ + D₂O)

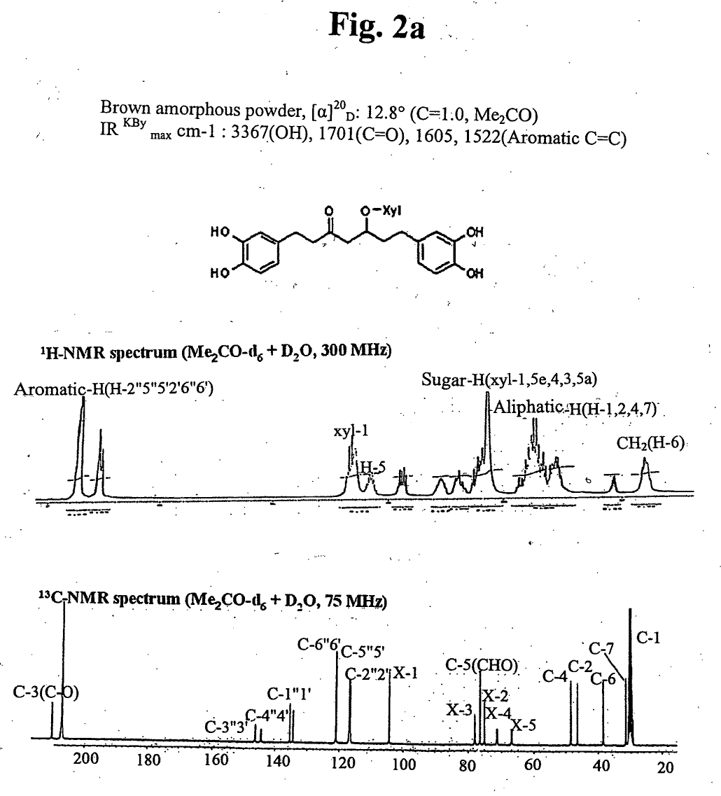

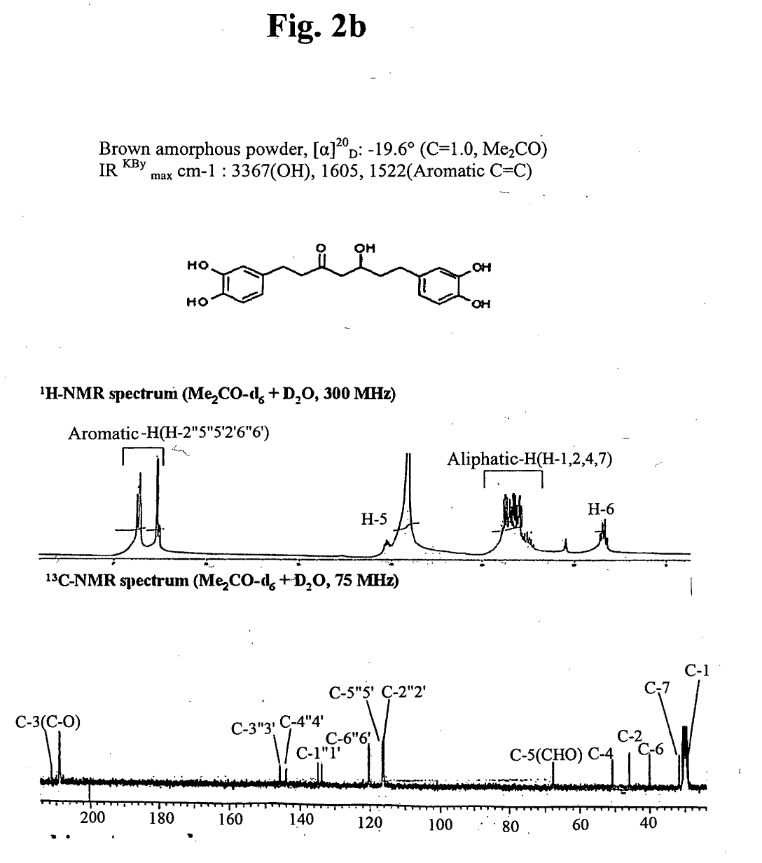

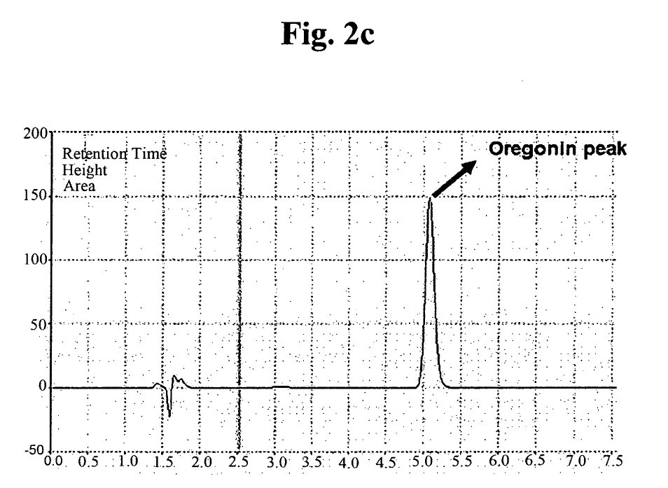

¹H-NMR and ¹³C-NMR spectra of oregonin were represented in FIG. 2a. ¹H-NMR and ¹³C-NMR spectra of hirsutanonol were represented in FIG. 2b. NMR and MS data of hirsutanonol and oregonin were consistent with the reference, identifying their structure.

### 5. Preparation of Active Ingredient Stock Solution

The hirsutanonol isolated as an active ingredient was not easily dissolved in water due to aglycone and so suspended in 10% DMSO at a concentration of 10 mg/ml. The final concentration of DMSO was adjusted to not more than 0.1% so as to maximally exclude the interference of the solvent. The oregonin isolated as the active ingredient was feasibly dissolved in water due to glycosides and so suspended in distilled water at a concentration of 10 mg/ml. The prepared stock solutions were stored and then used after dilution to the suitable concentration.

### 6. Cell Culture

The immune-related cell lines were cultured and then the immune regulatory activity of hirsutanonol and oregonin in vitro was determined in the cell line. Human T cell (Jurkat), rat mast cell (RBL-2H3), RAW264.7, HaCat and mouse splenocyte (Balb/c) were utilized and the culture of each cell line was carried out according to a conventional method and condition known to those skilled in the art.

### 7. Preparation of Formulation Containing Oregonin

7.1. HPLC Assay Condition and Calibration Curve Method

The stock solution (100 μg/ml) of oregonin purified from the stem bark of Alnus japonica was prepared by dissolving in methanol and the standard solutions of various concentrations of 1, 5, 10, 25, 50 μg/ml were fabricated by diluting the stock solution. The mixture of acetonitrile and 0.3% distilled water (70:30 v/v) was used for the mobile phase, and detection wavelength, injection volume and flow rate was 280 nm, 20 μl and 1 ml/min respectively. The retention time of oregonin was 5.1 min under the above conditions. In HPLC assay, calibration curve based on each concentration and peak area was made and represented in suitable linearity (R²=0.997) at a range of concentration of 1-50 μg/ml. HPLC assay of oregonin was shown in FIG. 2c.

7.2. Preparation of Injection Formulation Containing Oregonin

For evaluation of oregonin efficacy through injection, the aqueous injection formulations containing 0.1% or 1% (w/v) oregonin were prepared according to the compositions of the following Table 3. 10 and 100 mg of oregonin were weighed respectively and dissolved in suitable amounts of sterilized distilled water for injection. The pH of these solutions was adjusted to between pH 6.5 and pH 7.4 by adding small amount of NaOH solution and then fabricated to pH 10 by adding sterilized distilled water for injection. Formulation of drugs was determined using HPLC.

TABLE 3
Composition of Oregonin for Injection

| Ingredients | Formulation (%) |
|---|---|
| Oregonin | 0.1-1.0 |
| NaOH | q.s. |
| Water for injection | q.s. |
| Total | 100 |

7.3. Preparation of Ointment Formulation Containing Oregonin

The content of oregonin was fixed as 1% (w/w). 0/W (oil-in-water) cream ointment was prepared according to the following method. The compositions of 0/W cream were formulated according to component ratio of Table 4. Polyglyceryl-3-methylglucose distearate, stearic acid, cetyl alcohol and paraffin liquid were mixed depending on compositions and were formulated as oil phase by heating at 65° C. On the other hand, glycerin, distilled water and oregonin were mixed and completely dissolved by heating at 65° C., finally formulated as the aqueous phase. Oil solution was added to water solution at 65° C. and emulsified for several minutes using a homogenizer. After cooling, 0/W cream was formulated.

TABLE 4
Composition of Oregonin Ointment Formulation

| Ingredients | Formulation (%) |
|---|---|
| Polyglyceryl-3 methylglucose distearate | 3 |
| Stearic acid | 5 |
| Cetyl alcohol | 2 |
| Mineral oil | 7 |
| Glycerin | 10 |
| Water | 73 |

### 8. Animal Model of Atopic Dermatitis

8.1. Mite Patch

To examine treatment efficacy of oregonin on atopic dermatitis, the present inventors utilized an experimental animal model in which atopic dermatitis was induced by attachment of mite patch to mouse. The patch containing a mite-derived ingredient as a human atopic dermatitis-inducing agent was applied to 5-old-week mice. The hairs of their back were partially removed and mite patch was attached. At 2-week post-application, skin damage similar to atopic dermatitis was observed. Mite patch was detached at 18 weeks and drugs were administrated into mice.

8.2. NC/Nga Mouse

To research treatment efficacy of oregonin on atopic dermatitis, the inventors utilized NC/Nga mouse, an animal model known to those skilled in the art (Vestergaard C, Yoneyama H, Murai M, Nakamura K, Tamaki K, Terashima Y, Imai T, Yoshie O, Irimura T, Mizutani H and Matsushima K. Overproduction of Th2-specific chemokines in NC/Nga mice exhibiting atopic dermatitis-like lesions, J Clin Invest, 104 (8): 1097-105 (1999)).

### 9. Eosinophil Count

Mouse blood was collected into capillary tube and 30 μl of mouse whole blood was diluted 6-fold by addition of 150 μl saline. Eosinophiles were counted using a Sysmex XE-2100 hematology analyzer. Peripheral dormal slide was prepared and stained with Wright-Giemsa. Eosinophil count was carried out by differentially counting 200 leukocytes.

### 10. ELISA Measurement

Serum was prepared from blood in a main artery. Lymphocytes extracted from spleen were cultured. Briefly, extracted spleen was homogenized and filtered through mesh, isolating single cells. Red blood cells were lysed in RBC lysis buffer and the supernatant was removed by centrifugation. 1×10⁶ cells were cultured in 24-well plate containing RPMI media (1 ml). The experiments were performed in media containing cells cultured for 3 days.

### 11. Real-Time PCR

The primers used in Real-time PCR are as follows.

TABLE 5
Primer Sequences

| | |
|---|---|
| Primer | Sequence |

| | |
|---|---|
| MBD-1 (362bp) | 5′-ACATAAAGGACGAGCGATGG-3′ (sense) |
| | 5′-TGCAGATGGGGTGTCATAGA-3′ (anti-sense) |
| MBD-2 (199bp) | 5′-GCCATGAGGACTCTCTGCTC-3′ (sense) |
| | 5′-AGG GGT TCT TCT CTG GGA AA-3 (anti-sense) |
| MBD-3 (169bp) | 5′-TCA GAT TGG CAG TTG TGG AG-3′ (sense) |
| | 5′-GCT AGG GAG CAC TTG TTT GC-3′ (anti-sense) |
| iNOS (203bp) | 5′-CTG ATG CCT CTT CCA GGT GT-3′ (sense) |
| | 5′-GAG GGA GCC CTT TCT GAA TC-3′ (anti-sense) |
| COX-2 (593bp) | 5′-CCA CCC ATG GCA AAT TCC ATG GCA-3′ (sense) |
| | 5′-GGTGCTGCTTGTTAGGAGGTCAAGTAAAGGGC-3′(anti-sense) |
| GAPDH (598bp) | 5′-CCA CCC ATG GCA AAT TCC ATG GCA-3′ (sense) |
| | 5′-CCC TGT TGC TGT AGC CGT AT-3′ (anti-sense) |

Total RNA was extracted according to the following steps. Tissue was treated with 1 ml TRIZol reagent and mixed with 200 μl chloroform. After centrifugation at 12,000 rpm for 15 min at 4° C., the supernatant was transferred into a new tube and mixed with ½ vol. of isopropanol. The mixture was again centrifuged at 12,000 rpm for 15 min at 4° C. The supernatant was discarded and total RNA was dissolved in DEPC-water.

cDNA was prepared according to the following steps. The isolated total RNA was dissolved in 30 μl DEPC-DW and reverse transcription was performed using 3 μg of total RNA as a template in a reaction mixture (20 μl) containing 1 μl of reverse transcriptase, 2 μl of 10× buffer, 2 μl of 10 mM dNTP (dNTP mix), 1 μl of oligo dT primer, 0.5 μl of RNase inhibitor and 4 μl of 25 mM MgCl₂.

Each 2 μl of cDNA prepared was amplified using PCR. PCR reaction was performed by forty five cycles of 1 min at 59° C. and 1 min at 94° C. Finally, the mixtures were incubated at 72° C. for 1 min for extension.

### 12. Western Blotting

Skin tissue containing epidermal cells was lysed and centrifuged using a centrifuge. The supernatant was electrophoresized on 15% SDS-PAGE (sodium dodecyl sulfate polyacrylamide gel electrophoresis). For analysis of the expression levels of MBD-1, MBD-2, MBD-3, COX-2 and iNOS, proteins separated on gel electrophoresis were transferred into a nitrocellulose membrane and sequentially incubated with a primary antibody (1:1000 in BSA, goat polyclonal anti-MBD (mouse beta defensin) 1, 2 or 3 antibody, rabbit polyclonal anti-COX-2 antibody, rabbit polyclonal iNOS antibody; Chemicon, CA, USA) and a secondary antibody (1:2000 in BSA, anti-goat IgG, anti-rabbit IgG; Chemicon, CA, USA).

### Results

### 1. Assessment of In Vitro Activity of Oregonin

1.1. Cytotoxicity

Cytotoxicity of oregonin was carried out using LDH assay. LDH (lactate dehydrogenase) is a stable cytosolic enzyme released upon cell lysis. The measurement method of LDH is similar to that of ⁵¹Cr. In more detailed, LDH secreted reduces yellow tetrazolium salt to red formazan by LDH-coupled enzyme reaction and the absorbance measured is proportionate to cell number. Therefore, cytotoxicity of target substance could be indirectly determined by measuring the absorbance.

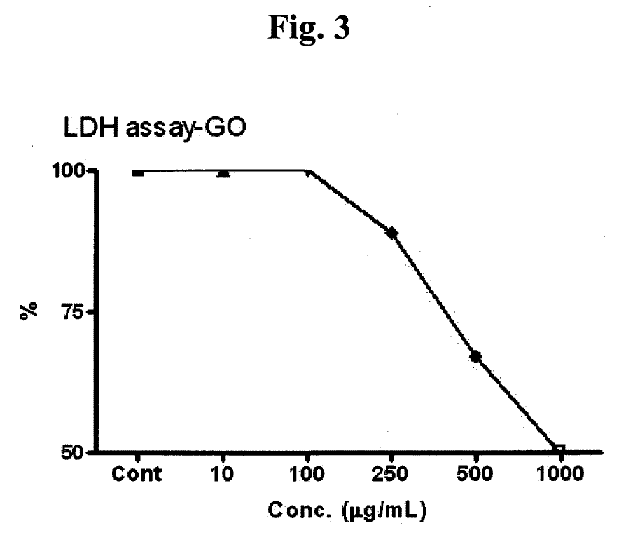

Cytotoxicity of oregonin measured by LDH assay was represented in FIG. 3. Oregonin solution, at a concentration range of 10-1000 μg/ml, was added to human T cells (Jurkat cells). In LDH assay of oregonin, it was demonstrated that cytotoxicity of 250 μg/ml oregonin was significantly lowered. The following experiments were performed using 250 μg/ml oregonin as a maximal effective amount.

1.2. NO Removal Activity

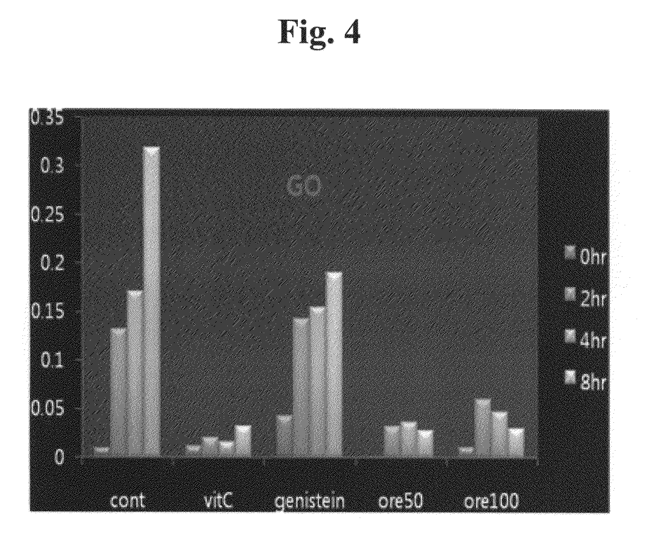

To indirectly examine anti-oxidative activity of oregonin, after SNAP (S-nitrosol-N-acetylpenicillamine) as nitric oxide (NO) donor and oregonin were treated together, NO removal activity of oregonin was measured by quantifying the amount of nitrogen dioxide produced over time. The amount of NO was quantified at predetermined point using Gries reagent and the effect between each groups was compared. Concretely, 1 mM SNAP, 50 μM Vitamin C and 1-500 μg/ml oregonin were treated and incubated at 37° C. for 0, 1, 2, 4 and 8 hrs, respectively. Griess reagent, a NO detection reagent, was added to each group and the absorbance at 540 nm was measured. Each value measured at predetermined point was calculated using a NO standard curve. Variation in the amount of NO generated of each group was measured at predetermined point. It could be appreciated that oregonin has much more excellent NO removal activity than Vitamin C and genistein, as shown in FIG. 4.

1.3. Anti-Oxidative Activity

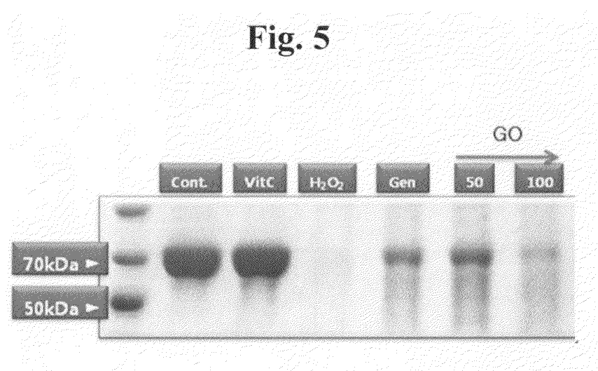

Anti-oxidative activity of oregonin was assessed. Evaluation of anti-oxidative activity was preformed according to a BSA (bovine serum albumin) degradation test. BSA degradation test is a method to verify a capacity of an anti-oxidative compound which protects proteins or enzymes from damage by reactive oxygen species (ROS) using a metal ion-catalyzed reaction. BSA (0.5 μg/mL) as a target protein, Cu²⁺ (100 μM) and H₂O₂ (2.5 mM) were mixed and then incubated with oregonins added in a concentration-dependent manner. The reaction mixture was electrophoresized on 10% SDS-PAGE (SDS-polyacrylamide gel). As a result, it was demonstrated that BSA degradation is inhibited by oregonin. Vitamin C (1 mM) with high anti-oxidative activity was used as a positive control. As shown in FIG. 5, remarkable activity of oregonin to inhibit BSA degradation was verified and it could be appreciated that oregonin has excellent anti-oxidative activity in the senses that final administration concentration of Vit. C is high concentration of 1 mM.

1.4. Regulatory Activity on Cytokine Expression

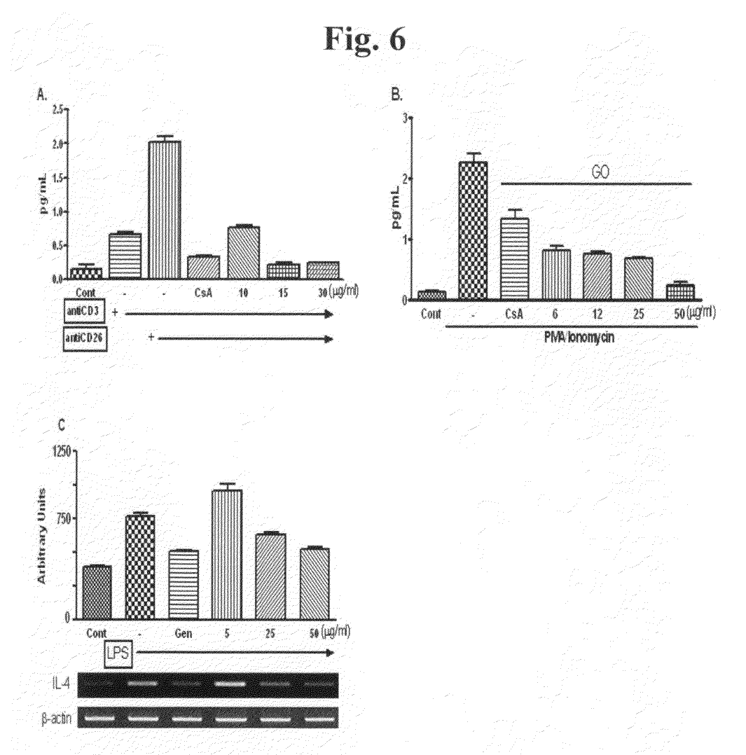

IL-4 (Th2 cytokine) was increased and IL-2 and IFN-γ (Th1 cytokine) was relatively decreased in patients with atopic dermatitis. The present inventors investigated whether oregonin has a regulatory ability of cytokines expressed in atopic dermatitis. Jurkat cell line (human T cells) was treated with oregonin and expression pattern of IL-2 as a Th1-secreted cytokine was measured using ELISA. In addition, regulation of IL-4 expression as a Th1 cytokine associated with atopic dermatitis was determined. As shown in FIG. 6, oregonin effectively inhibited IL-2 expression in TCR- and PMA/inomycin-activated T cells (panel A and B of FIG. 6). In addition, oregonin significantly prevented IL-4 expression (Th2 cytokine) in rat mast cells (RBL-2H3) (panel C of FIG. 6).

### 2. Assessment of In Vitro Activity of Hirsutanonol

2.1. Inhibitory Activity of Cell Proliferation

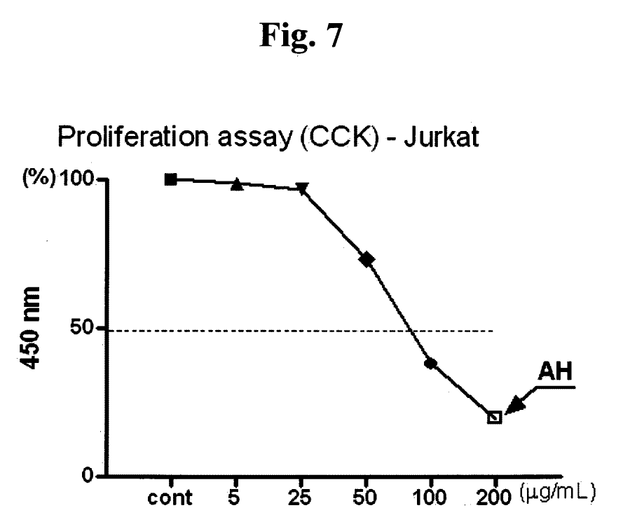

Hirsutanonol activity to inhibit cell proliferation of Jurkat cells was tested using CCK assay. As represented in FIG. 7, hirsutanonol exhibited inhibitory effects on cell proliferation at the concentration over 50 μg/ml. Therefore, about 50 μg/ml of hirsutanonol was used in experiments to assess in vitro activity of hirsutanonol.

2.2. Inhibitory Activity of Th1 Cytokine, IL-2 Expression

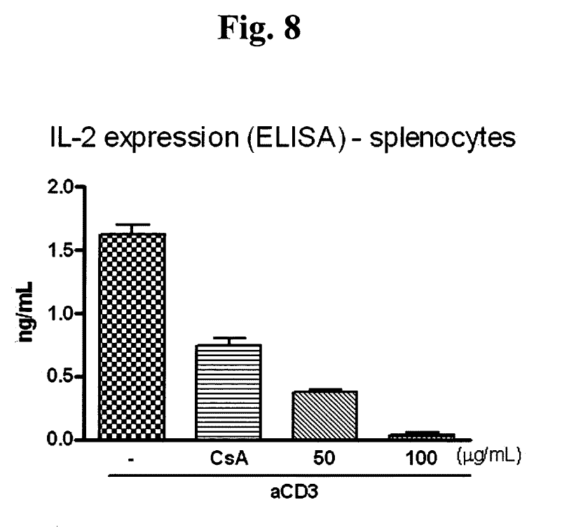

IL-2 is a cytokine expressed in Th1 cells and plays a crucial role in the proliferation of T cells. Hirsutanonol was added to splenic T cells which have been primarily cultured from murine splenocytes and pre-activated by anti-CD3 mAb. Inhibitory activity of hirsutanonol on IL-2 expression was measured using ELISA. Cyclosporine A (CsA) with strong inhibitory activity against IL-2 secretion was served as a positive control. The measurement results were shown in FIG. 8. It could be appreciated that the inhibitory effect of 50 μg/ml or 100 μg/ml hirsutanonol on IL-2 expression is much higher than that of CsA, as represented in FIG. 8. In particular, the inhibition effect of hirsutanonol on IL-2 secretion was remarkably exhibited at a concentration of 50 μg/ml without cytotoxicity.

2.3. Inhibitory Activity on Th1 and Th2-Cell Cytokine Expression

The inventors demonstrated regulatory activity of hirsutanonol on the cytokines expressed in Th cells which play an essential role in the development of acute and chronic atopic dermatitis. The mRNA expression levels of Th1 cytokines, IL-2 and Th2 cytokines, IL-4, IL-10 and IL-13 were examined. T cells activated by anti-CD3 mAb among spleen lymphocytes were used as a target. The primer sequences used to analyze IL-2, IL-4, IL-10 and IL-13 mRNA are as follows.

TABLE 6
Primer sequences

| Primer | Directions | Sequence |
|---|---|---|
| IL-2 | Sense | 5′-agatgaacttgcacctctgcg-3′ |
| | anti-sense | 5′-gggcttgttgagatgctttg-3′ |
| IL-4 | Sense | 5′-gtcactgacggcacagagcta-3′ |
| | anti-sense | 5′-ggactcattcatggtgcagctt-3′ |
| IL-10 | Sense | 5′-aagagagctccatcatgcctggct-3′ |
| | anti-sense | 5′-aatcgatgacagcgcctcagc-3′ |
| IL-13 | Sense | 5′-tcatggcgctctgggtgactgcag-3′ |
| | anti-sense | 5′-aggccaaagctgaggcatctccct-3′ |

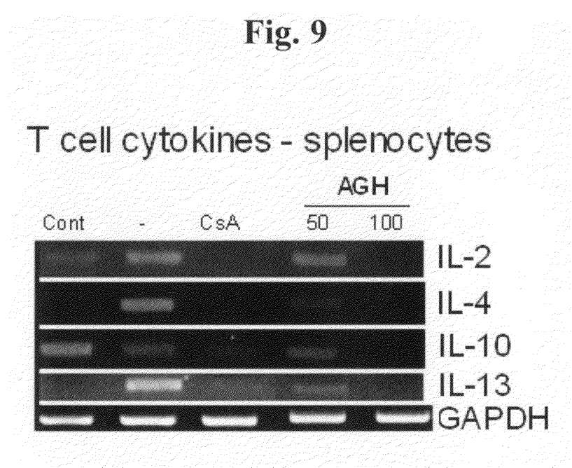

As shown in FIG. 9, hirsutanonol exhibited significant inhibitory activity on the mRNA expression of IL-2, IL-4, IL-10 and IL-13 at the concentration of 50 μg/ml or 100 μg/ml, and had inhibitory effect equal to CsA at the concentration of 100 μg/ml.

2.4. β-Hexosaminidase Assay

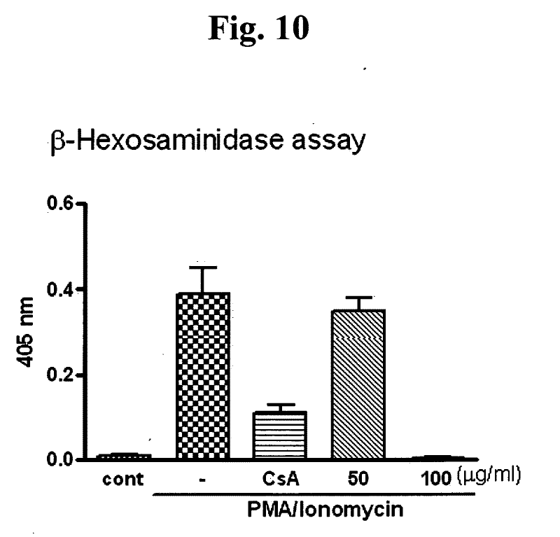

For testing the degranulation in rat mast cell (RBL-2H3) by hirsutanonol, β-hexosaminidase assay was carried out. β-hexosaminidase assay allows us to measure the activity and degranulation of mast cells. As shown in FIG. 10, the activity level of β-hexosaminidase was maximally inhibited at a concentration of 100 μg/ml hirsutanonol, suggesting that hirsutanonol has 20-fold inhibitory capability compared to CsA.

2.5. Anti-Oxidative Activity

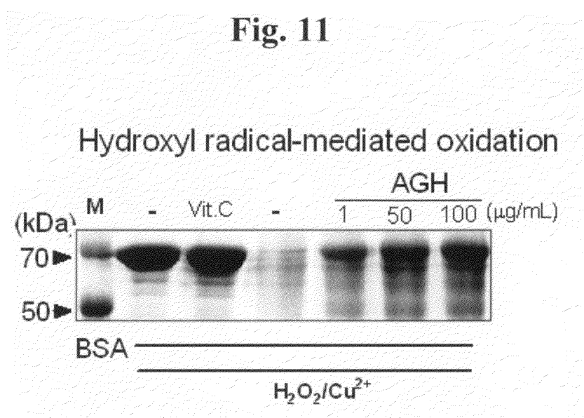

To examine anti-oxidative activity of hirsutanonol, the removal of radical produced from H₂O₂/Cu²⁺ was measured. Specifically, the anti-oxidative activity of hirsutanonol was determined by measuring the capability to inhibit the BSA protein degradation during the process of the BSA degradation by the action of the produced radical. As represented in FIG. 11, the activity of hirsutanonol to protect BSA was increased in a concentration-dependent manner, but protection effect of hirsutanonol was lower than that of Vitamin C used as a positive control. Nevertheless, hirsutanonol significantly exhibited BSA protection effect at a concentration of 100 μg/ml.

2.6. Inhibitory Activity on T-cell Activation Marker

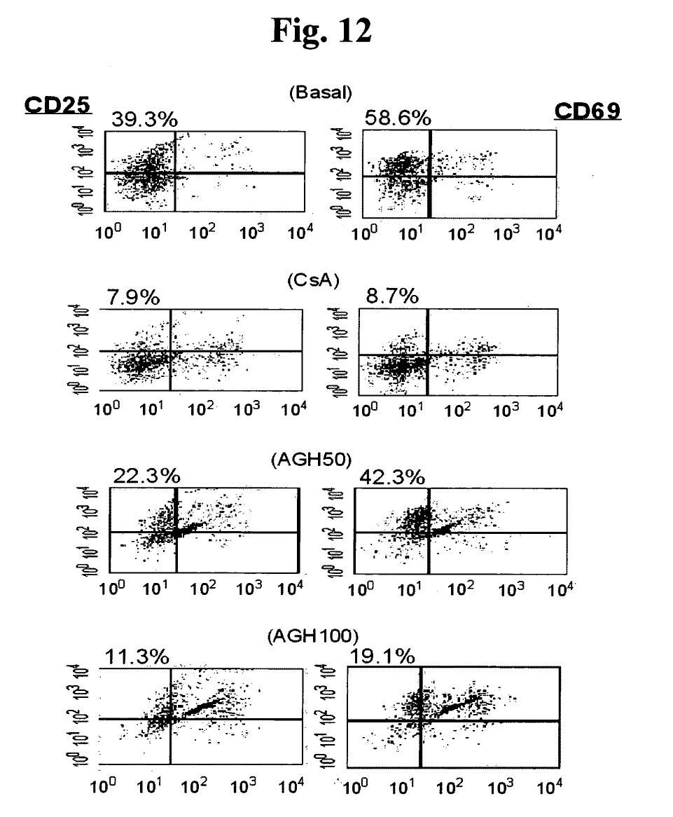

The activity of hirsutanonol to inhibit the expression of T-cell early activation markers (CD25 and CD69) was demonstrated by using a flow cytometry analysis (FACS). CD25 and CD69 expression levels were comparatively measured in the group of basal, CsA and hirsutanonol (50, 100 μg/ml) respectively. CsA was used as a control group due to its strong inhibitory activity on CD25 and CD69 expression. The results were represented in FIG. 12. 100 μg/ml hirsutanonol showed excellent inhibitory activity on CD25/CD69 expression compared to basal and CsA.

### 3. Assessment of In Vivo Activity of Oregonin

3.1. Naked-Eye Observation of Atopic Dermatitis in Animal Model

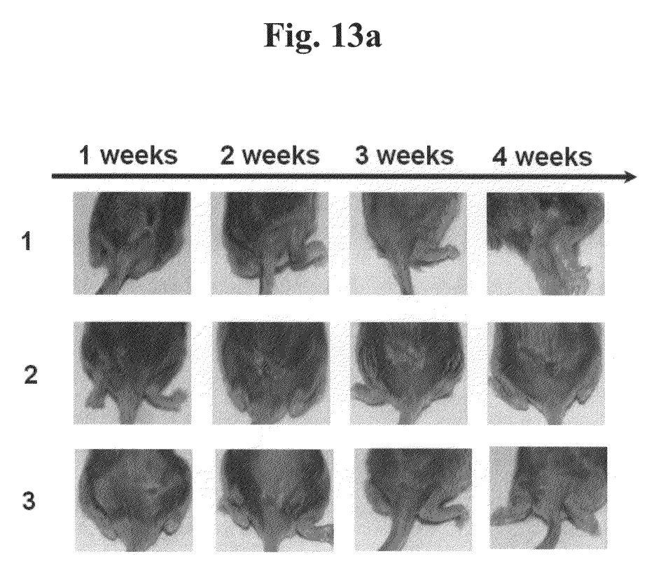

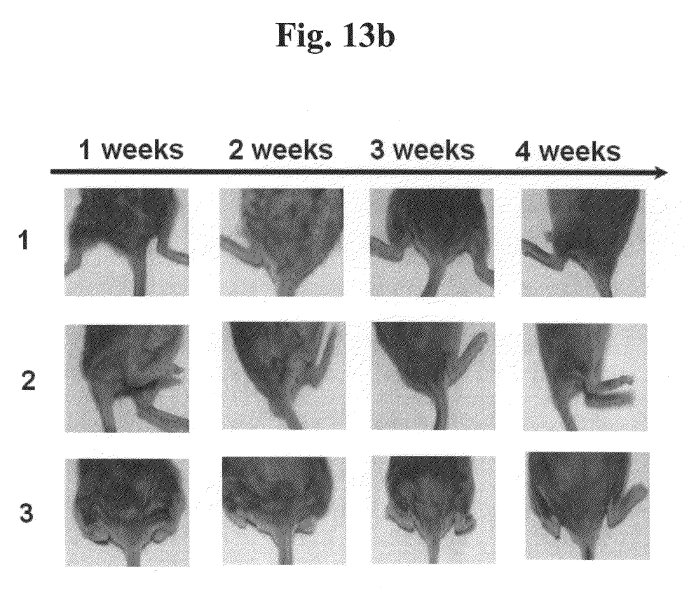

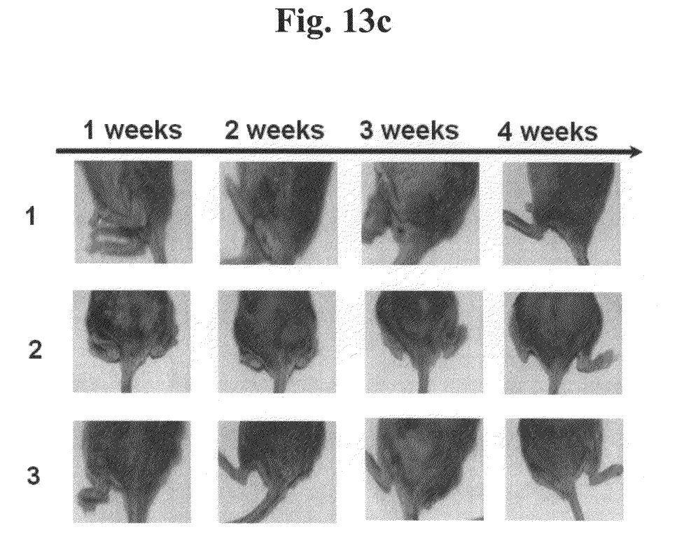

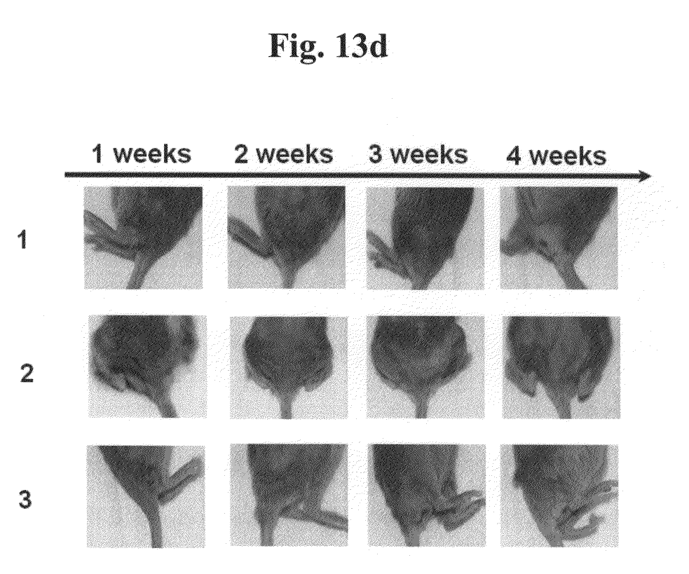

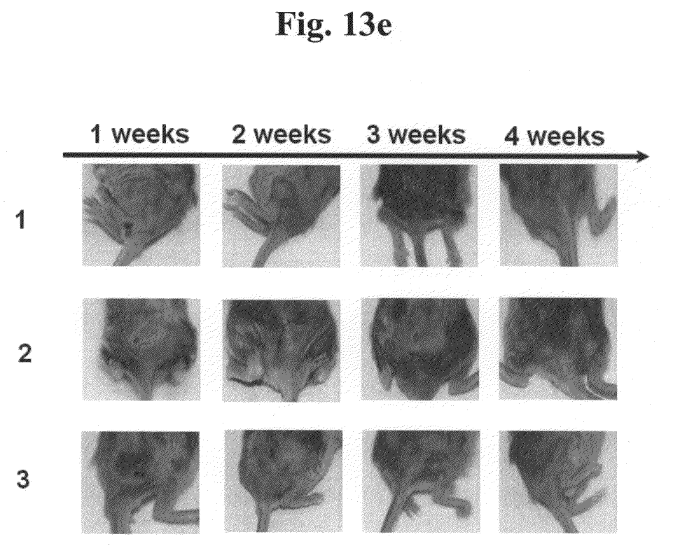

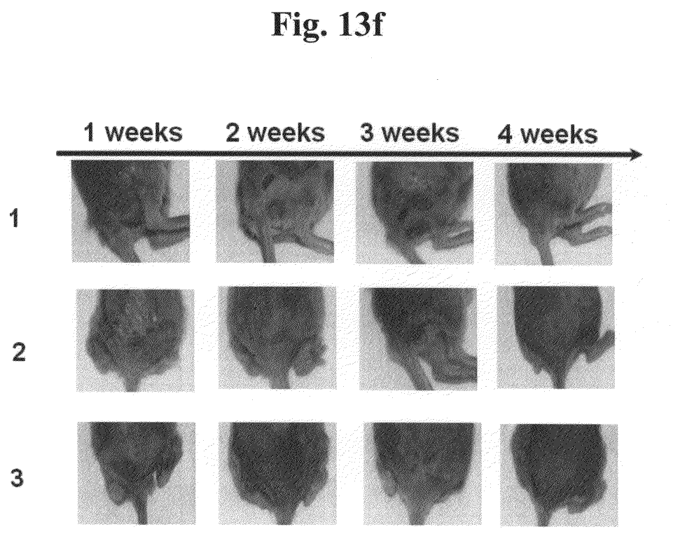

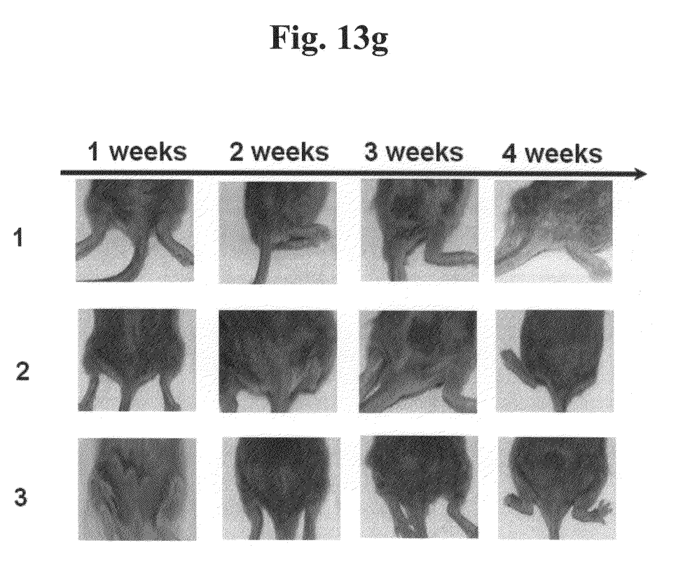

To examine treatment efficiency of oregonin on atopic dermatitis, the atopic dermatitis induced mouse was treated with oregonin by administration via peritoneal or dermal application for 4 weeks. The assessment of external skin condition of atopic dermatitis lesion was made by naked-eye observation. As a result, the skin conditions of the group treated with oregonin were improved compared to the negative control group. That is, it was demonstrated that atopic dermatitis was significantly relieved in mice in which 0.1% and 1% oregonin (each FIG. 13b and FIG. 13c) was administrated through an injection compared to the mice in which PBS (FIG. 13a) was administrated as a negative control. In addition, it was also demonstrated that atopic dermatitis was much more alleviated in mice in which 1% oregonin (FIG. 13f) was applied to skin using an ointment than in mice treated with a composition not containing oregonin (FIG. 13e). FIG. 13g is photographs showing treatment effects on atopic dermatitis after plancol was applied on the skin of the atopic dermatitis induced mice as a positive control.

3.2. Eosinophil Count

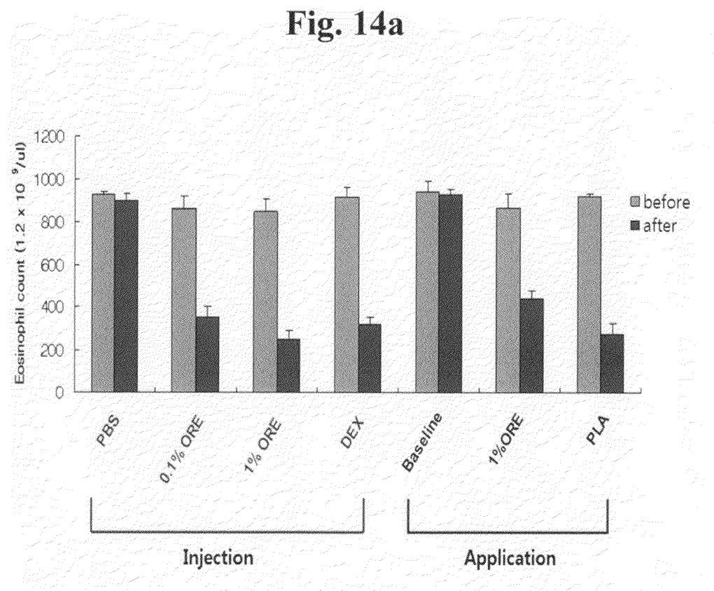

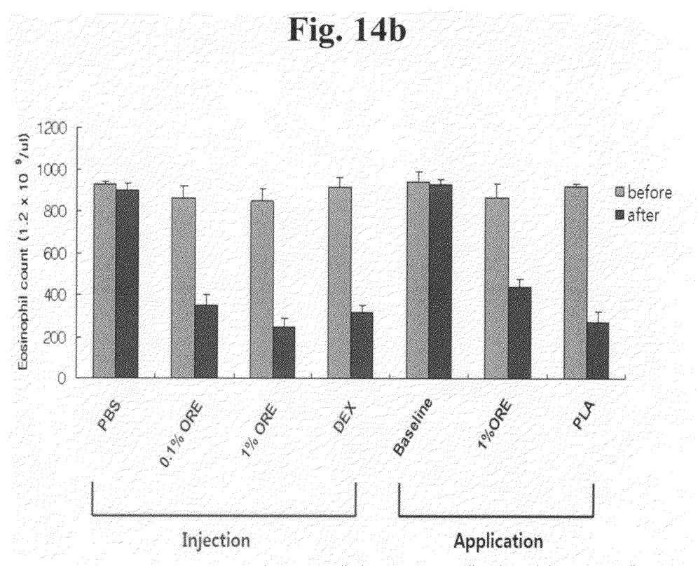

NC/Nga mice in which atopic dermatitis was induced were treated with oregonin by administration via peritoneal or dermal application for 4 weeks. Blood was collected from the mice before and after administration or application and then the number of eosinophil was measured. It could be appreciated that the number of eosinophil after administration or application of oregonin was more severely reduced than that before administration or application of oregonin (FIGS. 14a-14b).

3.3. Measurement of IL (Interleukin)-4, IL-5, IL-10 and IL-13 Expression Level

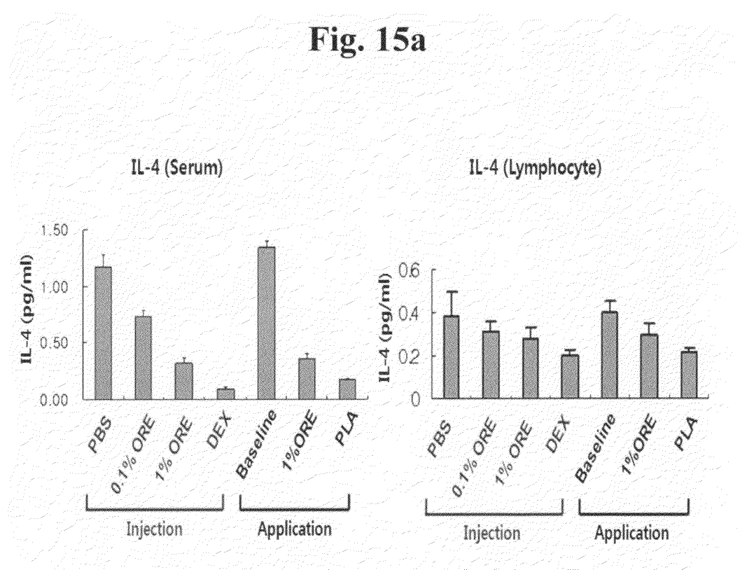

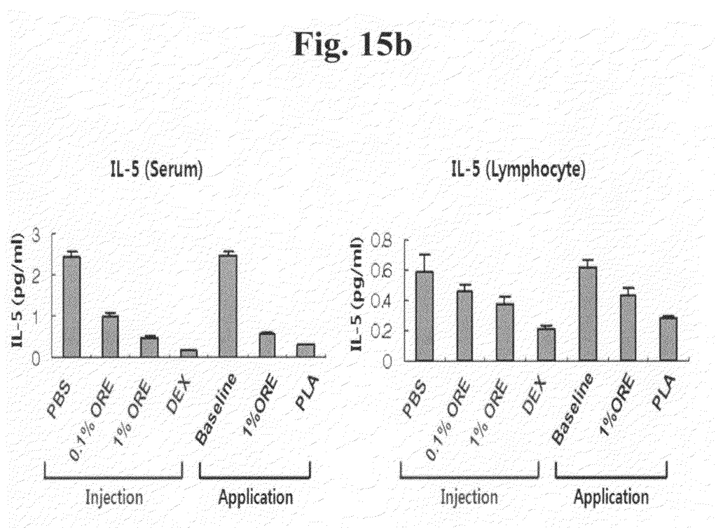

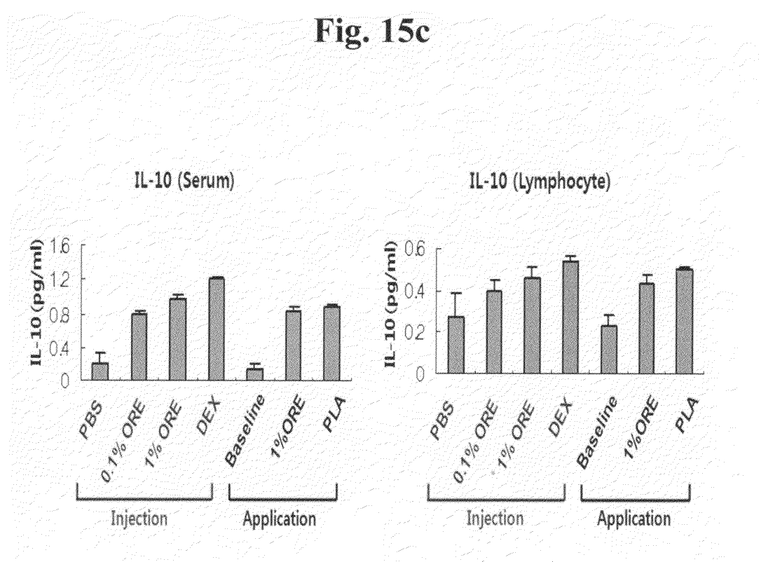

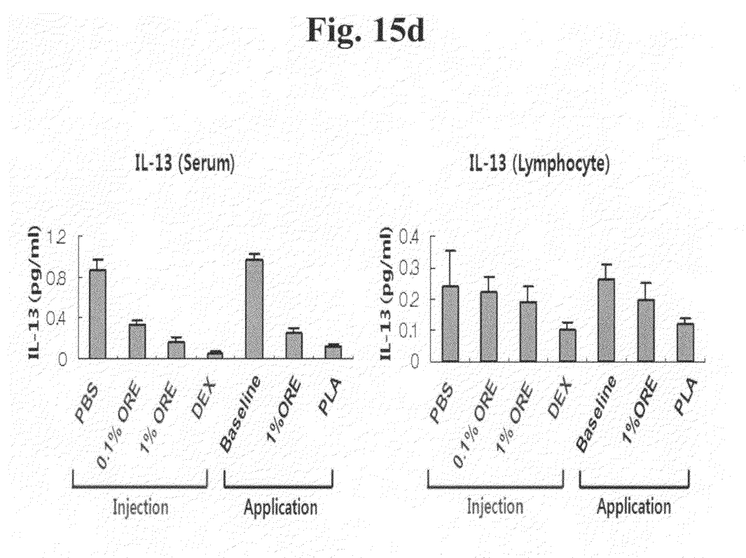

Atopic dermatitis induced NC/Nga mice were treated with oregonin by administration via peritoneal or dermal application for 4 weeks. Blood and spleen were collected from mice and expression levels of IL (interleukin)-4, IL-5, IL-10 and IL-13 in serum and splenocyte were measured using ELISA. IL-4, IL-5 and IL-13 expression levels detected in group of oregonin administration or application were lower than that of negative control group (FIG. 15a, FIG. 15b, FIG. 15d). However, IL-10 expression level was more increased in groups of oregonin administration or application than that of negative control (FIG. 15c).

IL-4 promotes cell proliferation of activated B cells and T cells and cell differentiation of naïve T cell (CD4⁺ T cell) towards Th2 cell. IL-4 expression level was increased in patients with atopic dermatitis. It could be appreciated that oregonin of the present invention plays inhibitory role in allergy response by reduction of IL-4 expression.

It has been known that IL-5 accelerates secretion of immunoglobulin by stimulating growth of B cells and acts as a major mediator of eosinophil activation. It was demonstrated that oregonin of the present invention significantly decreases the number of eosinophil in the subjects with atopic dermatitis by reducing IL-5 expression.

IL-10 is an anti-inflammatory cytokine known as human cytokine synthesis inhibitory factor (CSIF) and inhibits synthesis of pro-inflammatory cytokines such as IFN-γ, IL-2, IL-3, TNF-α and GM-CSF produced in macrophage and T helper cell type 1 (Th1). It was demonstrated that oregonin of the present invention increases IL-10 level, exhibiting anti-inflammation effect.

IL-13 is secreted in various cell types, particularly T helper cell type 2 (Th2) and plays a critical role as a mediator in allergic inflammation diseases. It was demonstrated that oregonin of this invention reduces IL-13 expression, representing valuable treatment efficacy of allergic inflammation diseases.

3.4. Measurement of MBD-1, MBD-2, MBD-3, COX-2 and iNOS Expression Using Real-time PCR

Atopic dermatitis induced NC/Nga mice were treated with oregonin by administration via peritoneal or dermal application for 4 weeks, and mice epidermal cells were collected. To investigate whether oregonin has a regulatory activity on immune response, the levels of MBD-1, MBD-2, MBD-3, COX-2 and iNOS expression were estimated using real-time PCR.

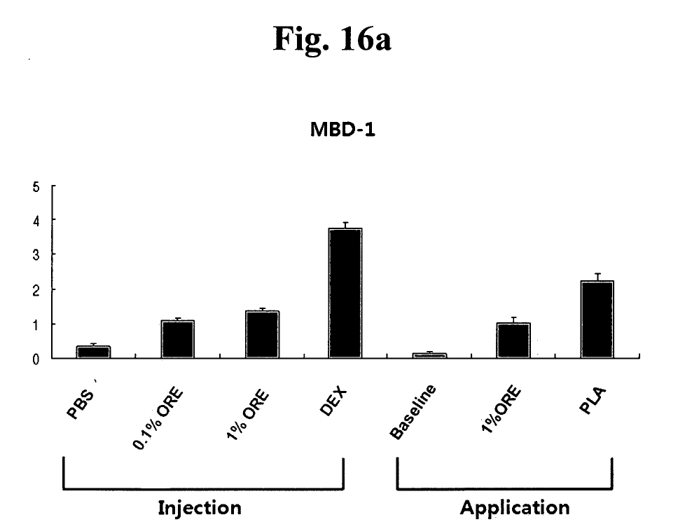

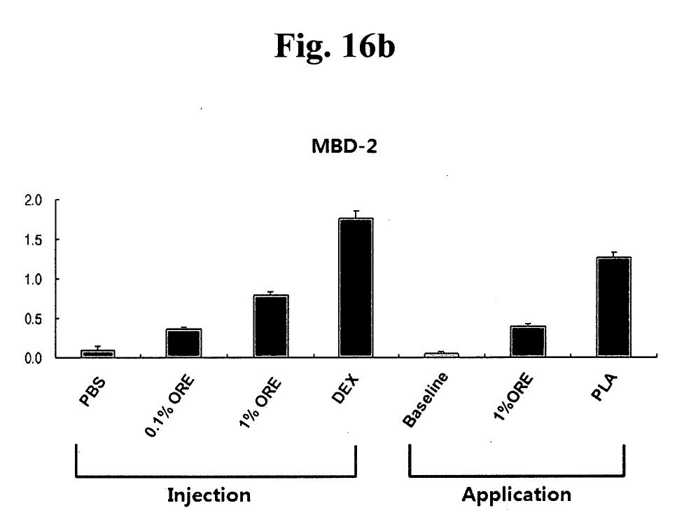

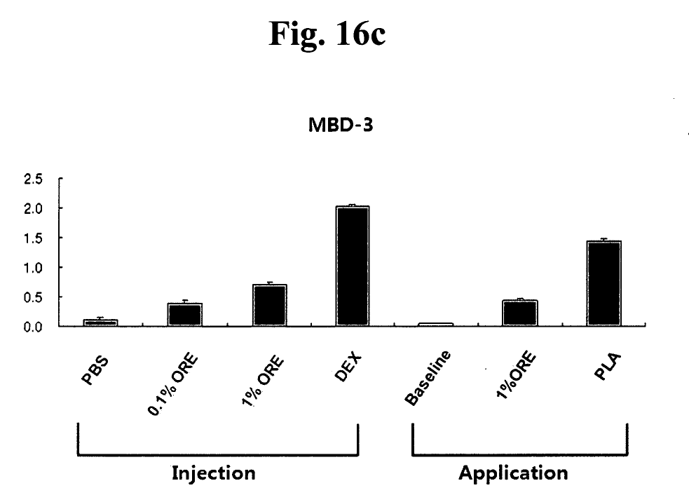

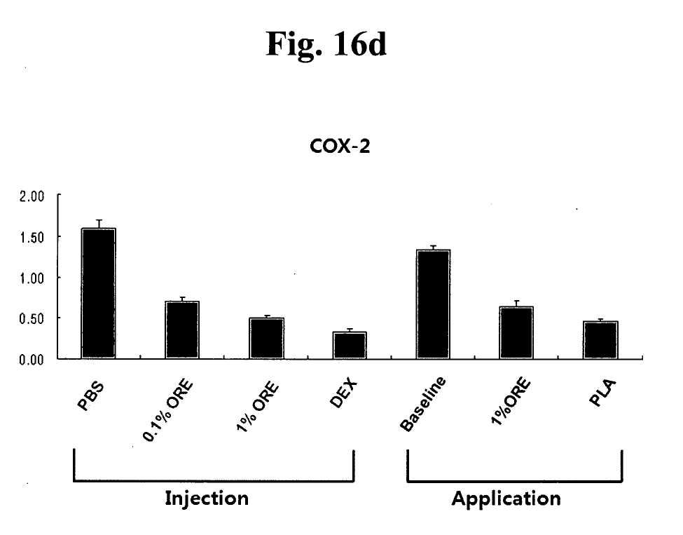

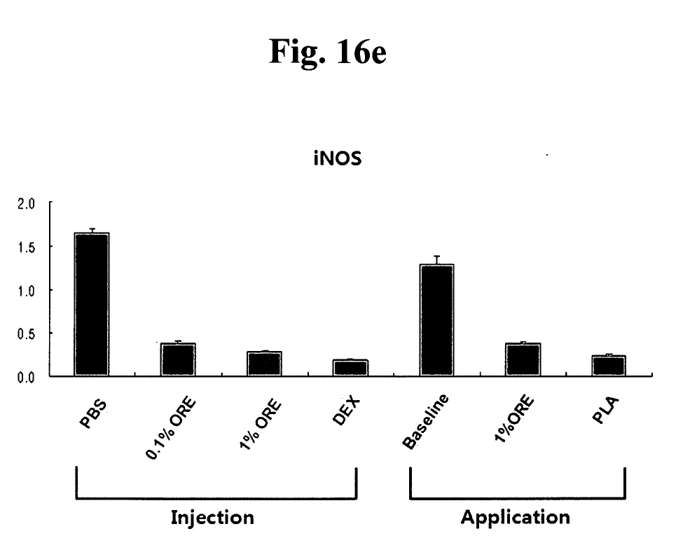

It is known that MBD (mouse beta-defensin) is mainly expressed in epithelial cells of epidermis, bronchus and urogenital organ and promotes release of histamine and prostaglandin D2 by stimulating mast cells. In addition, MBD is known to prevent bacterial growth in epithelial cells. COX (cyclooxygenase) is an enzyme associated with formation of biological mediators called prostanoids. It is known that COX-1 is related to maintenance of homeostasis of human body, while COX-2 is associated with immune response. In addition, iNOS (inducible Nitric Oxide Synthase) is an enzyme to produce NO (nitrix oxide) which regulates macrophage to play an important role in initial immune response against microorganism invasion. As results, it was demonstrated that the levels of MBD-1, MBD-2 and MBD-3 expression were increased in mice treated with oregonin (FIGS. 16a-16c), while the expression levels of COX-2 and iNOS were decreased (FIGS. 16d-16e).

3.5. Measurement of MBD-1, MBD-2, MBD-3, COX-2 and iNOS Expression Level Using Western Blotting

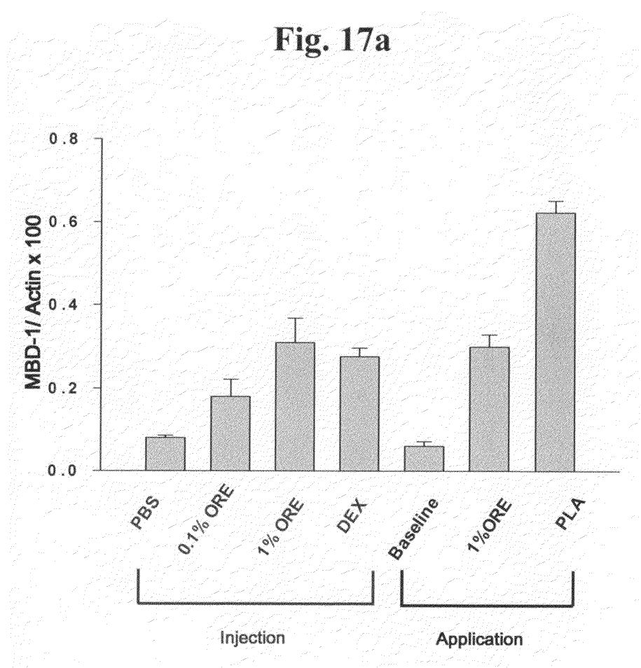

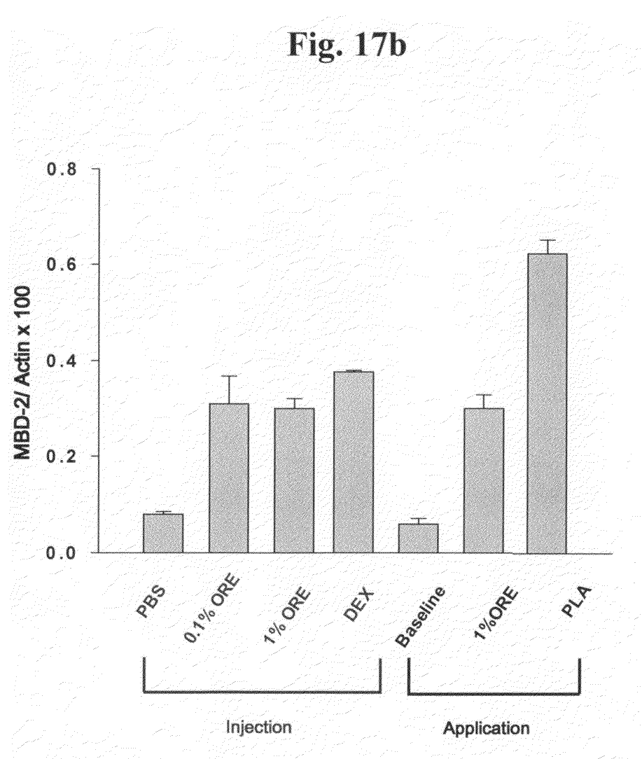

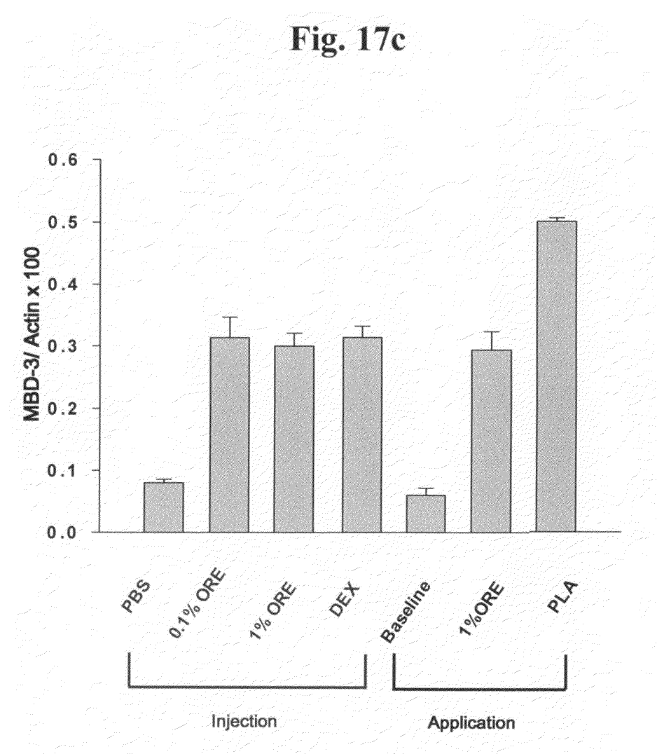

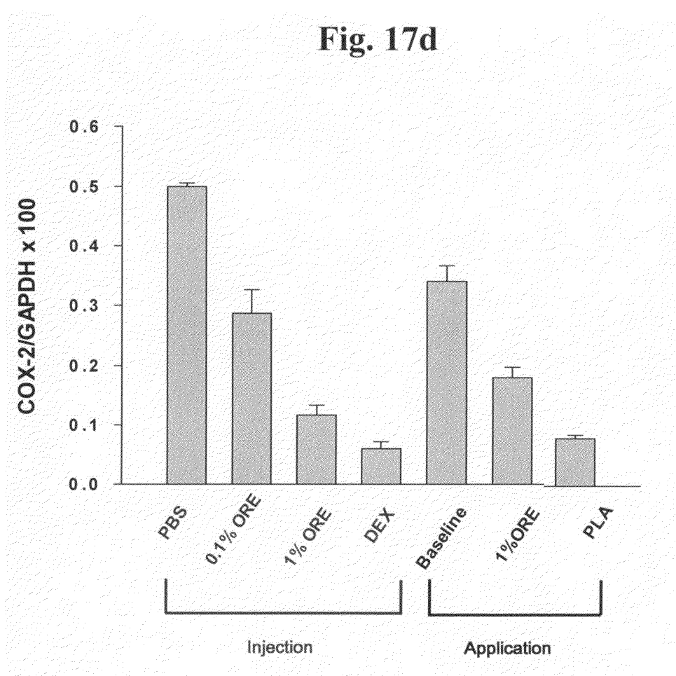

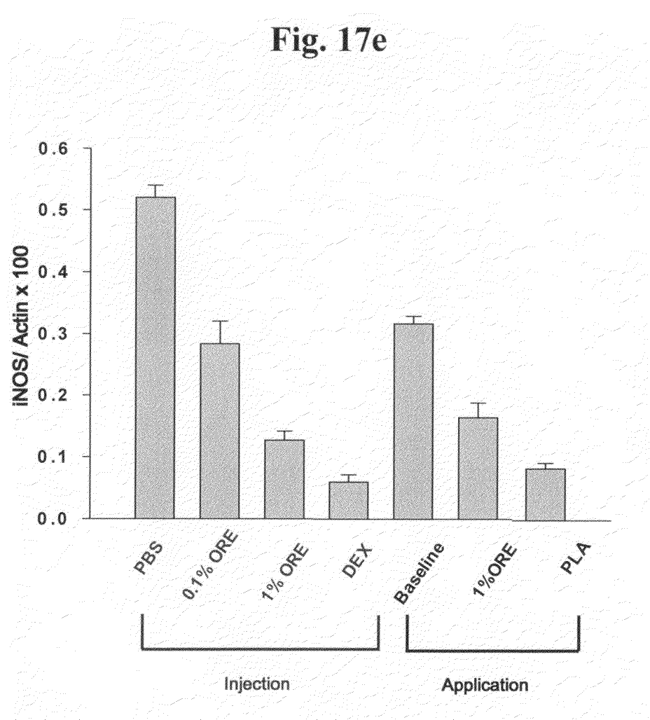

Atopic dermatitis induced NC/Nga mice were treated with oregonin liposomes by administration via peritoneal or dermal application for 4 weeks, and mice epidermal cells were collected. To investigate a regulatory activity of oregonin on immune response, the expression levels of MBD-1, MBD-2, MBD-3, COX-2 and iNOS were detected using western blotting. It was demonstrated that the expression levels of MBD-1, MBD-2, MBD-3 COX-2 and iNOS were reduce in mice treated with oregonin (FIGS. 17a-17e) as same as the above-mentioned results using real-time PCR.

As described above, it could be appreciated that hirsutanonol and oregonin of the present invention improve or treat atopic dermatitis by regulation of immune response associated with atopic dermatitis.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A pharmaceutical composition for treating or preventing atopic dermatitis comprising (a) a therapeutically effective amount of an isolated compound represented by the following formula 1 or 2; and (b) a pharmaceutically acceptable carrier. 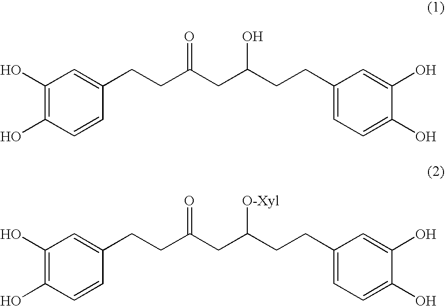
wherein ‘Xyl’ represents xylose in formula 2.

2. A cosmetic composition for relieving a symptom of atopic dermatitis comprising (a) a cosmetically effective amount of an isolated compound represented by the following formula 1 or 2; and (b) a cosmetically acceptable carrier. 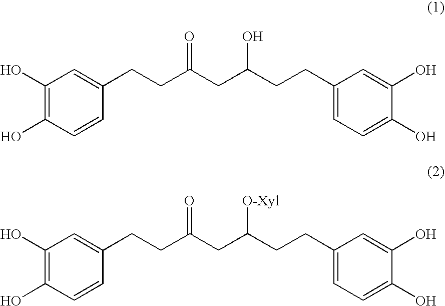
wherein ‘Xyl’ represents xylose in the formula 2.

3. A functional food composition for relieving a symptom of atopic dermatitis comprising an isolated compound represented by the following formula 1 or 2 as an active ingredient. 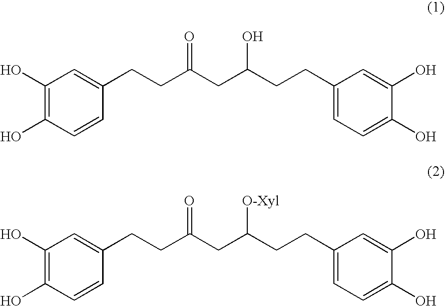
wherein ‘Xyl’ represents xylose in the formula 2.

4. A method for treating atopic dermatitis in a subject suffering from atopic dermatitis, which comprises contacting said subject with a composition comprising a therapeutically effective amount of an insolated compound represented by the following formula 1 or 2 or administering said composition to said subject. 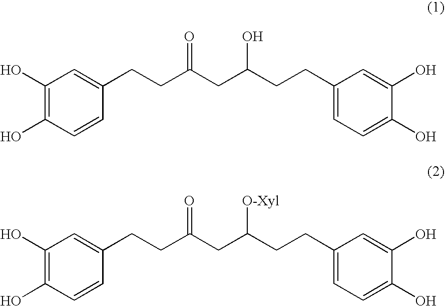
wherein ‘Xyl’ represents xylose in formula 2.

5. A method for relieving a symptom of atopic dermatitis in a subject suffering from atopic dermatitis, which comprises contacting said subject with a composition comprising an insolated compound represented by the following formula 1 or 2 or administering said composition to said subject. 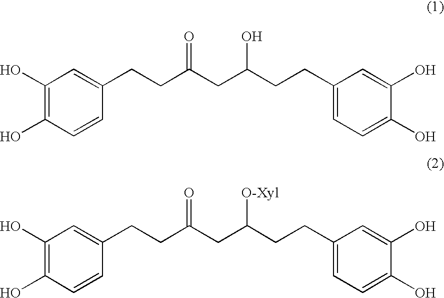
wherein ‘Xyl’ represents xylose in formula 2.

6. The method according to claim 5, wherein the composition is a cosmetic composition comprising a cosmetically effective amount of an isolated compound represented by the formula 1 or 2.

7. The method according to claim 5, wherein the composition is a functional food composition comprising an isolated compound represented by the formula 1 or 2.

8. The method according to claim 5, wherein the composition induces a reduction of eosinophil number, IgE level, or expression level of immune regulatory cytokine IL-4, IL-5 or IL-13.
